(19) United States
(12) Patent Application Publication
(10) Pub. No.: US 2015/0166470 A1
(43) Pub. Date: Jun. 18, 2015

(54) 7-DISUBSTITUTED-PHENYL TETRACYCLINE DERIVATIVES

(71) Applicant: Paratek Pharmaceuticals, Inc., Boston, MA (US)

(72) Inventors: Todd Bowser, Charlton, MA (US); Paul Abato, Providence, RI (US)

(73) Assignee: Paratek Pharmaceuticals, Inc., Boston, MA (US)

(21) Appl. No.: 14/403,616

(22) PCT Filed: May 30, 2013
(86) PCT No.: PCT/US13/43363

§ 371 Date: Nov. 25, 2014

Related U.S. Application Data
(60) Provisional application No. 61/653,262, filed on May 30, 2012.

(51) Int. Cl. C07C 237/34

(52) U.S. Cl. CPC C07C 237/34; C07C 2103/46

(57) ABSTRACT

7-Disubstituted-phenyl tetracycline compounds are disclosed herein. Also disclosed is a method for treatment or prevention of spinal muscular atrophy using the 7-disubstituted-phenyl tetracycline compounds. This invention also relates to a method for treating or preventing a subject having spinal muscular atrophy. The method includes administering to the subject an effective amount of a tetracycline compound of formula (1), such that the spinal muscular atrophy is treated or prevented. Advantageously, the tetracycline compounds used in this method of the invention have one or more of the following characteristics: 1) potency in modulating mRNA splicing, 2) potency in modulating SMN protein levels, 3) central nervous system (CNS) and/pr brain penetration, 4) decreased phototoxic properties and 5) decreased antibacterial properties.

### CROSS-REFERENCE TO RELATED APPLICATIONS

This application claims the benefit of priority from U.S. Provisional Application No. 61/653,262, filed May 30, 2012, the entire contents of which are hereby incorporated herein by reference in their entirety.

### BACKGROUND OF THE INVENTION

Spinal muscular atrophy (SMA) is a life-threatening disorder resulting from the absence of, or mutation in, the survival motor neuron 1 gene (SMN1). SMN1 is responsible for producing SMN protein (SMNp), which is necessary for survival of motor neurons in the spinal cord and the brain. In SMA patients, the expression of insufficient levels of SMN protein causes motor neuron degeneration and subsequent system-wide muscle wasting (atrophy). See Gilliam T. C. et al., Nature 345, 823-825 (1990).

SMN2 is almost identical to SMN1, but has a critical C-to-T transition in exon 7. This C-to-T transition alters splicing such that the resulting SMN2 mRNA lacks exon 7 (SMND7 mRNA) and consequently produces a truncated protein with reduced stability. See Lorson C. L. et al., Proc. Natl Acad. Sci. USA 96(11), 6307-6311 (1999). Nevertheless, approximately 10% of SMN2 transcripts are full length and produce functional SMN protein. It has been found that the clinical severity of SMA is inversely related to the SMN2 copy number. See Feldkotter M. et al., Am. J. Hum. Genet. 70(2), 358-368 (2002).

To date, SMA is still the leading genetic cause of death in infants and toddlers and afflicts 1 in 6,000-10,000 children born. Therefore, there is a need for effective therapy for this disease.

### SUMMARY OF THE INVENTION

The invention relates to a compound of formula (I) or a pharmaceutically acceptable salt thereof:

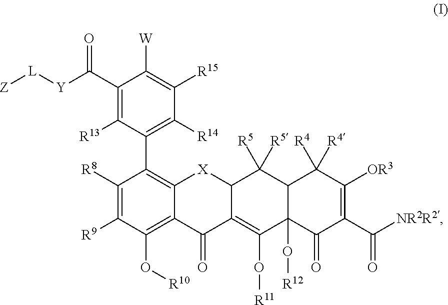

wherein

X is CR⁶R^{6′}, C═CR⁶R^{6′}, NR^{6″}, O, or S;

each of R², R^{2′}, and R^{6″}, independently, is hydrogen, halo, or Rⁱ, Rⁱ being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkoxy, C₁-C₆ alkylcarbonyl, arylcarbonyl, C₁-C₆ alkylsulfinyl, arylsulfinyl, C₁-C₆ alkylsulfonyl, arylsulfonyl, or arylalkyl;

each of R³, R¹⁰, R¹¹ and R¹², independently, is hydrogen or Rⁱⁱ, Rⁱⁱ being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkylcarbonyl, arylcarbonyl, C₁-C₆ alkylsulfinyl, arylsulfinyl, C₁-C₆ alkylsulfonyl, arylsulfonyl, or arylalkyl;

each of R⁴, R^{4′}, R⁵, R^{5′}, R⁶, R^{6′}, R⁸, R⁹, R¹³, R¹⁴, and R¹⁵, independently, is hydrogen, halo, nitro, cyano, hydroxyl, thiol, or Rⁱⁱⁱ, Rⁱⁱⁱ being C₁-C₆ alkyl, C₂-C₆ alkenyl, C₂-C₆ alkynyl, aryl, heteroaryl, C₃-C₈ cycloalkyl, 3 to 8-membered heterocycloalkyl, arylalkyl, C₁-C₆ alkyloxy, C₃-C₈ cycloalkyloxy, 3 to 8-membered heterocycloalkyloxy, aryloxy, heteroaryloxy, C₁-C₆ alkylthio, arylthio, C₁-C₆ alkylsulfinyl, arylsulfinyl, C₁-C₆ alkylsulfonyl, arylsulfonyl, C₁-C₆ alkylamino, arylamino, di-C₁-C₆ alkylamino, diarylamino, C₁-C₆ alkylcarbonyl, C₃-C₈ cycloalkylcarbonyl, 3 to 8-membered heterocycloalkylcarbonyl, arylcarbonyl, heteroarylcarbonyl, C₁-C₆ alkylcarboxyl, C₃-C₈ cycloalkylcarboxyl, 3 to 8-membered heterocycloalkylcarboxyl, arylcarboxyl, heteroarylcarboxyl, C₁-C₆ alkyloxycarbonyl, C₃-C₈ cycloalkyloxycarbonyl, 3 to 8-membered heterocycloalkyloxycarbonyl, aryloxycarbonyl, heteroaryloxycarbonyl, C₁-C₆ alkylcarbamido, C₃-C₈ cycloalkylcarbamido, 3 to 8-membered heterocycloalkylcarbamido, arylcarbamido, heteroarylcarbamido, C₁-C₆ alkylcarbamyl, C₃-C₈ cycloalkylcarbamyl, 3 to 8-membered heterocycloalkylcarbamyl, arylcarbamyl, or heteroarylcarbamyl;

W is halo, alkyl, amino, cyano, nitro, C₁-C₆ alkoxy, hydroxyl, or thiol;

Y is O, or NR^{7a};

L is C₁₋₆ alkylene;

Z is OR^{7b}, SR^{7b}, or NR^{7b}R^{7c}; and

each of R^{7a}, R^{7b}, and R^{7c}, independently, is hydrogen, halo, or R^{iv}, R^{iv} being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkoxy, C₁-C₆ alkylcarbonyl, arylcarbonyl, C₁-C₆ alkylsulfinyl, arylsulfinyl, C₁-C₆ alkylsulfonyl, arylsulfonyl, or arylalkyl. Each of Rⁱ, Rⁱⁱ, Rⁱⁱⁱ, and R^{iv} mentioned above is optionally substituted with one or more substituents selected from the group consisting of halogen, cyano, nitro, hydroxyl, amino, thiol, C₁-C₆ alkyl, C₂-C₆ alkenyl, C₂-C₆ alkynyl, aryl, heteroaryl, C₃-C₈ cycloalkyl, 3 to 8-membered heterocycloalkyl, arylalkyl, C₁-C₆ alkyloxy, C₃-C₈ cycloalkyloxy, 3 to 8-membered heterocycloalkyloxy, aryloxy, heteroaryloxy, C₁-C₆ alkylthio, arylthio, C₁-C₆ alkylsulfinyl, arylsulfinyl, C₁-C₆ alkylsulfonyl, arylsulfonyl, C₁-C₆ alkylamino, arylamino, di-C₁-C₆ alkylamino, diarylamino, C₁-C₆ alkylcarbonyl, C₃-C₈ cycloalkylcarbonyl, 3 to 8-membered heterocycloalkylcarbonyl, arylcarbonyl, heteroarylcarbonyl, C₁-C₆ alkylcarboxyl, C₃-C₈ cycloalkylcarboxyl, 3 to 8-membered heterocycloalkylcarboxyl, arylcarboxyl, heteroarylcarboxyl, C₁-C₆ alkyloxycarbonyl, C₃-C₈ cycloalkyloxycarbonyl, 3 to 8-membered heterocycloalkyloxycarbonyl, aryloxycarbonyl, heteroaryloxycarbonyl, C₁-C₆ alkylcarbamido, C₃-C₈ cycloalkylcarbamido, 3 to 8-membered heterocycloalkylcarbamido, arylcarbamido, heteroarylcarbamido, C₁-C₆ alkylcarbamyl, C₃-C₈ cycloalkylcarbamyl, 3 to 8-membered heterocycloalkylcarbamyl, arylcarbamyl, and heteroarylcarbamyl.

This invention also relates to a method for treating or preventing a subject having spinal muscular atrophy. The method includes administering to the subject an effective amount of a tetracycline compound of formula (I), such that the spinal muscular atrophy is treated or prevented. Advantageously, the tetracycline compounds used in this method of the invention have one or more of the following characteristics: 1) potency in modulating mRNA splicing, 2) potency in modulating SMN protein levels, 3) central nervous system (CNS) and/or brain penetration, 4) decreased phototoxic properties and 5) decreased antibacterial properties.

This invention also relates to a method for modulating SMN2 mRNA splicing. The method includes contacting SMN2 mRNA with a tetracycline compound of formula (I), such that SMN2 mRNA splicing is modulated.

This invention also relates to a method for modulating SMNp levels in a subject in need thereof by administering to the subject an effective amount of a tetracycline compound of formula (I), such that SMNp levels are modulated in the subject.

This invention also relates to a pharmaceutical composition containing a tetracycline compound of formula (I) and a pharmaceutically acceptable carrier. Such a pharmaceutical composition can be used in treating or preventing SMA or modulating SMN2 mRNA splicing and SMNp levels.

Further, the invention relates to a packaged tetracycline compound, comprising an effective amount of a tetracycline compound and instructions for using the tetracycline compound for the treatment or prevention of spinal muscular atrophy.

### BRIEF DESCRIPTION OF THE DRAWINGS

FIG. 1 is a chart illustrating in vitro correction of cell-free splicing of SMN2 mRNA by compound 1.

FIG. 2 is a graph illustrating the ratios of full length/truncated mRNA in SMN2 splicing in SMA patient cells after treated with Compound 1 at different concentratons.

FIG. 3 is a graph illustrating the increase of SMN protein levels in SMA patient cells treated in vitro with Compound 1 at different concentrations.

FIG. 4 is a graph showing the pharmacokinetics of Compound 1 in mice.

FIG. 5 is a chart illustrating the increase of Exon 7 inclusion in the brain tissue of neonatal transgenic mice treated with Compound 1.

FIG. 6 is a graph illustrating survival time of Compound 1-treated severe SMA mice vs. that of untreated severe SMA mice.

FIG. 7 is a graph illustrating survival curve of SMA mice following administration of Compound 1.

### DETAILED DESCRIPTION OF THE INVENTION

This invention pertains, at least in part, to tetracycline compounds having formula (I) shown below:

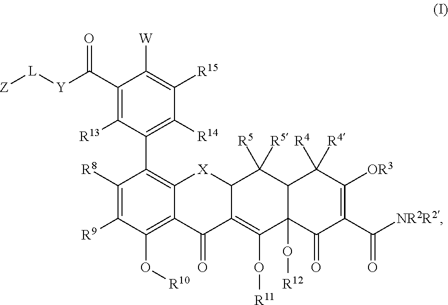

wherein

X is CR⁶R^{6′}, C═CR⁶R^{6′}, NR^{6″}, O, or S;

each of R², R^{2′}, and R^{6″}, independently, is hydrogen, halo, or Rⁱ, Rⁱ being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkoxy, C₁-C₆ alkylcarbonyl, arylcarbonyl, C₁-C₆ alkylsulfinyl, arylsulfinyl, C₁-C₆ alkylsulfonyl, arylsulfonyl, or arylalkyl;

each of R³, R¹⁰, R¹¹, and R¹², independently, is hydrogen or Rⁱⁱ, Rⁱⁱ being C₁-C₆ alky, C₂-C₆ alkenyl, C₂-C₆ alkynyl, C₃-C₈ cycloalkyl, 3 to 8-membered heterocycloalkyl, aryl, heteroaryl, C₁-C₆ alkylcarbonyl, arylcarbonyl, C₁-C₆ alkylsulfinyl, arylsulfinyl, C₁-C₆ alkylsulfonyl, arylsulfonyl, arylalkyl, or a prodrug moiety;

each of R⁴, R^{4′}, R⁵, R^{5′}, R⁶, R^{6′}, R⁸, R⁹, R¹³, R¹⁴, and R¹⁵, independently, is hydrogen, halo, nitro, cyano, hydroxyl, thiol, or Rⁱⁱⁱ, Rⁱⁱⁱ being C₁-C₆ alkyl, C₂-C₆ alkenyl, C₂-C₆ alkynyl, aryl, heteroaryl, C₃-C₈ cycloalkyl, 3 to 8-membered heterocycloalkyl, arylalkyl, C₁-C₆ alkyloxy, C₃-C₈ cycloalkyloxy, 3 to 8-membered heterocycloalkyloxy, aryloxy, heteroaryloxy, C₁-C₆ alkylthio, arylthio, C₁-C₆ alkylsulfinyl, arylsulfinyl, C₁-C₆ alkylsulfonyl, arylsulfonyl, C₁-C₆ alkylamino, arylamino, di-C₁-C₆ alkylamino, diarylamino, C₁-C₆ alkylcarbonyl, C₃-C₈ cycloalkylcarbonyl, 3 to 8-membered heterocycloalkylcarbonyl, arylcarbonyl, heteroarylcarbonyl, C₁-C₆ alkylcarboxyl, C₃-C₈ cycloalkylcarboxyl, 3 to 8-membered heterocycloalkylcarboxyl, arylcarboxyl, heteroarylcarboxyl, C₁-C₆ alkyloxycarbonyl, C₃-C₈ cycloalkyloxycarbonyl, 3 to 8-membered heterocycloalkyloxycarbonyl, aryloxycarbonyl, heteroaryloxycarbonyl, C₁-C₆ alkylcarbamido, C₃-C₈ cycloalkylcarbamido, 3 to 8-membered heterocycloalkylcarbamido, arylcarbamido, heteroarylcarbamido, C₁-C₆ alkylcarbamyl, C₃-C₈ cycloalkylcarbamyl, 3 to 8-membered heterocycloalkylcarbamyl, arylcarbamyl, or heteroarylcarbamyl;

W is halo, alkyl, amino, cyano, nitro, C₁-C₆ alkoxy, hydroxyl, or thiol;

Y is O, or NR^{7a};

L is C₁₋₆ alkylene;

Z is OR^{7b}, SR^{7b}, or NR^{7b}R^{7c}; and

each of R^{7a}, R^{7b}, and R^{7c}, independently, is hydrogen, halo, or R^{iv}, R^{iv} being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkoxy, C₁-C₆ alkylcarbonyl, arylcarbonyl, C₁-C₆ alkylsulfinyl, arylsulfinyl, C₁-C₆ alkylsulfonyl, arylsulfonyl, or arylalkyl. Each of Rⁱ, Rⁱⁱ, Rⁱⁱⁱ, and R^{iv} mentioned above is optionally substituted with one or more substituents selected from the group consisting of halogen, cyano, nitro, hydroxyl, amino, thiol, C₁-C₆ alkyl, C₂-C₆ alkenyl, C₂-C₆ alkynyl, aryl, heteroaryl, C₃-C₈ cycloalkyl, 3 to 8-membered heterocycloalkyl, arylalkyl, C₁-C₆ alkyloxy, C₃-C₈ cycloalkyloxy, 3 to 8-membered heterocycloalkyloxy, aryloxy, heteroaryloxy, C₁-C₆ alkylthio, arylthio, C₁-C₆ alkylsulfinyl, arylsulfinyl, C₁-C₆ alkylsulfonyl, arylsulfonyl, C₁-C₆ alkylamino, arylamino, di-C₁-C₆ alkylamino, diarylamino, C₁-C₆ alkylcarbonyl, C₃-C₈ cycloalkylcarbonyl, 3 to 8-membered heterocycloalkylcarbonyl, arylcarbonyl, heteroarylcarbonyl, C₁-C₆ alkylcarboxyl, C₃-C₈ cycloalkylcarboxyl, 3 to 8-membered heterocycloalkylcarboxyl, arylcarboxyl, heteroarylcarboxyl, C₁-C₆ alkyloxycarbonyl, C₃-C₈ cycloalkyloxycarbonyl, 3 to 8-membered heterocycloalkyloxycarbonyl, aryloxycarbonyl, heteroaryloxycarbonyl, C₁-C₆ alkylcarbamido, C₃-C₈ cycloalkylcarbamido, 3 to 8-membered heterocycloalkylcarbamido, arylcarbamido, heteroarylcarbamido, C₁-C₆ alkylcarbamyl, C₃-C₈ cycloalkylcarbamyl, 3 to 8-membered heterocycloalkylcarbamyl, arylcarbamyl, and heteroarylcarbamyl.

In one embodiment, R⁴ is NR^{4a}R^{4b} and R^{4′} is H; wherein each of R^{4a} and R^{4b} is independently H, C₁-C₆ alkyl, C₁-C₆ alkylcarbonyl, arylcarbonyl, C₁-C₆ alkylsulfinyl, arylsulfinyl, C₁-C₆ alkylsulfonyl, arylsulfonyl, or arylalkyl. In a further embodiment, each of R^{4a} and R^{4b} is C₁-C₆ alkyl (e.g., methyl).

In another embodiment, one of R³, R¹⁰, R¹¹, and R¹² is a prodrug moiety, e.g., a C₁₋₆ alkylcarbonyl. For example, R³ is a prodrug moiety, R¹⁰ is a prodrug moiety, R¹¹ is a prodrug moiety, or R¹² is a prodrug moiety. In a further embodiment, two or more of R³, R¹⁰, and R¹¹ are prodrug moieties.

In another embodiment, the compounds have one or more of the following features: X is CR⁶R⁶; R⁴ is NR^{4a}R^{4b}; R^{4a} and R^{4b} are each C₁-C₆ alkyl (e.g., methyl), and each of R², R^{2′}, R³, R^{4′}, R⁵, R^{5′}, R⁶, R^{6′}, R⁸, R⁹, R¹⁰, R¹¹, R¹², R¹³, R¹⁴, and R¹⁵ is hydrogen. For example, the compounds have all of these features and, thus, are of the following formula (II):

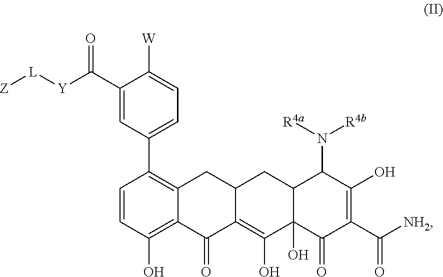

wherein W, Y, L, and Z are defined above.

The above formula (II), containing chiral centers, represents compounds that occur as a stereoisomeric mixture or a single stereoisomer. For example, the compounds may have a stereochemistry shown in the following formula (III) or (IV):

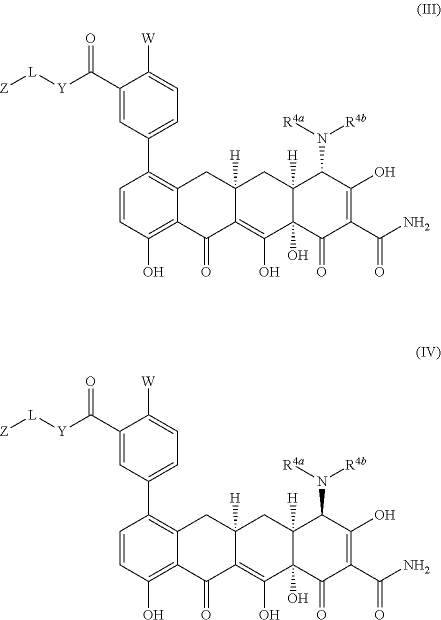

In another embodiment, the compounds of formula (I) have one or more of the following features: X is C═CR⁶R^{6′}; R⁴ is NR^{4a}R^{4b}; each of R^{4a} and R^{4b} is C₁-C₆ alkyl (e.g., methyl), and each of R², R^{2′}, R³, R^{4′}, R⁵, R^{5′}, R⁶, R^{6′}, R⁸, R⁹, R¹⁰, R¹¹, R¹², R¹³, R¹⁴, and R¹⁵ is hydrogen. For example, the compounds have all of the listed features. In a further embodiment, Y is NR^{7a}, R^{a} being H. In a further embodiment, L is C₂₋₃ alkylene (e.g., —CH₂CH₂—). In still a further embodiment, Z is OR^{7b}, R^{7b} being C₁-C₆ alkyl (e.g., methyl or ethyl). Alternatively, Z is NR^{7b}R^{7c}, each of R^{7b} and R^{7c}, independently, being hydrogen or C₁-C₆ alkyl (e.g., methyl). In yet a further embodiment, W is F, Cl, or I. For example, W is F.

In another embodiment, the compounds of formula (I) have one or more of the following features: X is C═CR⁶R^{6′}; R⁴ is NR^{4a}R^{4b}; R^{4a} and R^{4b} are each C₁-C₆ alkyl (e.g., methyl), and each of R², R^{2′}, R³, R^{4′}, R⁵, R^{5′}, R⁸, R⁹, R¹⁰, R¹¹, R¹², R¹³, R¹⁴, and R¹⁵ is hydrogen; R⁶ is methyl; and R^{6′} is hydroxyl. For example, the compounds have all of the listed features.

In another embodiment, the compounds of formula (I) have one or more of the following features: X is C═CR⁶R^{6′}; R⁴ is NR^{4a}R^{4b}; R^{4a} and R^{4b} are each C₁-C₆ alkyl (e.g., methyl), and each of R², R^{2′}, R³, R^{4′}, R⁵, R^{5′}, R^{6′}, R⁸, R⁹, R¹⁰, R¹¹, R¹², R¹³, R¹⁴, and R¹⁵ is hydrogen; and R⁶ is methyl. For example, the compounds have all of the listed features.

In another embodiment, referring to formulae (I), (II), (III), and (IV), Y is NR^{7a}, R^{a} being H. In a further embodiment, L is C₂₋₃ alkylene (e.g., —CH₂CH₂—). In still a further embodiment, Z is OR^{7b}, R^{7b} being C₁-C₆ alkyl (e.g., methyl or ethyl) or NR^{7b}R^{7c}, each of R^{7b} and R^{7c}, independently, being hydrogen or C₁-C₆ alkyl (e.g., methyl). In yet a further embodiment, W is F, Cl, or I. For example, W is F.

In another embodiment, referring to formulae (I), (II), (III), and (IV), W is not C₁-C₆ alkoxy. W can be halo, alkyl, amino, cyano, nitro, hydroxyl, or thiol.

In another embodiment, referring to formulae (I), (II), (III), and (IV), W is F, Cl, or I. For example, W is F. In a further embodiment, Y is NR^{7a}, R^{a} being H. In a further embodiment, L is C₂₋₃ alkylene (e.g., —CH₂CH₂—). In still a further embodiment, Z is OR^{7b}, R^{7b} being C₁-C₆ alkyl (e.g., methyl or ethyl) or NR^{7b}R^{7c}, each of R^{7b} and R^{7c}, independently, being hydrogen or C₁-C₆ alkyl (e.g., methyl).

In another embodiment, referring to formulae (I), (II), (III), and (IV), Z is OR^{7b}, R^{7b} being H or C₁-C₆ alkyl (e.g., methyl or ethyl).

In another embodiment, referring to formulae (I), (II), (III), and (IV), Z is NR^{7b}R^{7c}, each of R^{7b} and R^{7c}, independently, being hydrogen or C₁-C₆ alkyl (e.g., methyl). As an example, Z is N(CH₃)₂.

In another embodiment, referring to formulae (I), (II), (III), and (IV), L is straight or branched alkylene. For example, L is —CH₂—, —CH₂CH₂—, —CH₂CH₂CH₂—, or —CH₂CH₂CH₂CH₂—.

In another embodiment, referring to formulae (I), (II), (III), and (IV), R⁸ is H.

In another embodiment, referring to formulae (I), (II), (III), and (IV), R⁹ is H.

In another embodiment, referring to formulae (I), (II), (III), and (IV), each of R³, R¹⁰, and R¹¹ is H.

An exemplary compound of formula (I) is shown below:

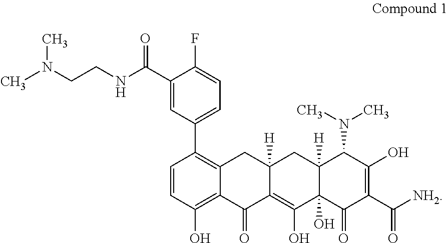 [SMILES: CN(C)CCNC(=O)c1cc(-c2ccc(O)c3c2C[C@H]2C[C@H]4[C@H](N(C)C)C(O)=C(C(N)=O)C(=O)[C@@]4(O)C(O)=C2C3=O)ccc1F]

The term “alkyl” refers to a monovalent straight or branched hydrocarbon. Examples of straight-chain alkyl groups include, but are not limited to, methyl, ethyl, propyl, butyl, pentyl, hexyl, heptyl, octyl, nonyl, and decyl. Examples of branched-chain alkyl groups include, but are not limited to, isopropyl, tert-butyl, and isobutyl. Unless stated otherwise, a alkyl group contains 1-20 carbon atoms in its backbone for straight chain and 3-20 carbon atoms for branched chain. The term “alkylene” refers to a bivalent straight or branched hydrocarbon, containing 1-20 carbon atoms. Examples of alkylene include, but are not limited to, methylene, ethylene, and propylene. The term “alkenyl” refers to a monovalent straight or branched hydrocarbon containing 2-20 carbon atoms and one or more double bonds. Examples of alkenyl, but are not limited to, include ethenyl, propenyl, allyl, and 1,4-butadienyl. The term “alkenylene” refers to a bivalent straight or branched hydrocarbon containing 2-20 carbon atoms and one or more double bonds. The term “alkynyl” refers to a monovalent straight or branched hydrocarbon containing 2-20 carbon atoms and one or more triple bonds. Examples of alkynyl include, but are not limited to, ethynyl, 1-propynyl, 1- and 2-butynyl, and 1-methyl-2-butynyl. The term “alkynylene” refers to a bivalent straight or branched hydrocarbon containing 2-20 carbon atoms and one or more triple bonds. The term “alkoxy” refers to an —O-alkyl radical. Examples of alkoxy include, but are not limited to, methoxy, ethoxy, n-propoxy, isopropoxy, n-butoxy, iso-butoxy, sec-butoxy, and tert-butoxy.

The term “cycloalkyl” refers to a monovalent saturated hydrocarbon ring system having 3 to 20 carbon atoms. Examples of cycloalkyl include, but are not limited to, cyclopropyl, cyclobutyl, cyclopentyl, cyclopentenyl, cyclohexyl, cyclohexenyl, cycloheptyl, and cyclooctyl.

The term “heterocycloalkyl” refers to cycloalkyl moieties in which one or more carbons of the cycloalkyl scaffold is replace with a heteroatom, for example, oxygen, nitrogen, sulfur or phosphorous. Examples of heterocyclic moieties include piperidine, morpholine, pyrrolidine, piperazine and tetrahydrofuran.

The term “aryl” refers to a monovalent 6-carbon monocyclic, 10-carbon bicyclic, 14-carbon tricyclic aromatic ring system. Examples of aryl groups include, but are not limited to, phenyl, naphthyl, and anthracenyl. The term “heteroaryl” refers to a monvalent aromatic 5-8 membered monocyclic, 8-12 membered bicyclic, or 11-14 membered tricyclic ring system having one or more heteroatoms (such as O, N, or S). Examples of heteroaryl groups include pyridyl, furyl, imidazolyl, benzimidazolyl, pyrimidinyl, thienyl, quinolinyl, indolyl, and thiazolyl.

The term “amino” refers to a nitrogen radical that is covalently bonded to two moieties selected from hydrogen, alkyl, aryl, cycloalkyl, heterocycloalkyl, and heteroaryl. The term includes “alkylamino” moieties, wherein the nitrogen is bound to at least one alkyl group. The term also includes “dialkylamino” groups wherein the nitrogen atom is bound to two alkyl groups. The term “arylamino” and “diarylamino” include groups wherein the nitrogen is bound to one or two aryl groups, respectively. The term “alkylarylamino,” “alkylaminoaryl” or “arylaminoalkyl” refers to an amino group which is bound to at least one alkyl group and at least one aryl group.

The term “carbonyl” refers to moieties which contain a carbon connected with a double bond to an oxygen atom. Examples of moieties which contain a carbonyl group include aldehydes, ketones, carboxylic acids, amides, esters, anhydrides, etc.

The term “carboxyl” includes moieties containing a carbonyl group, in which the carbon of the carbonyl group is covalently bound to two moieties: (i) an oxygen radical and (ii) a group selected from hydrogen, alkyl, aryl, cycloalkyl, heterocycloalkyl, and heteroaryl.

The term “halogen” includes fluorine, bromine, chlorine, and iodine. The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O⁻X⁺, where X⁺ is a counterion.

The term “sulfonyl” includes moieties which comprise a sulfonyl (S(═O)₂) group. Similarly, the term “sulfinyl” includes moieties which comprise a sulfinyl (S(═O)) group.

The term “carbamido” refers to amino-carbonyl (N—C(═O)) groups, in which the amino is covalently bound to two moieties selected from hydrogen, alkyl, aryl, cycloalkyl, heterocycloalkyl, and heteroaryl.

The term “carbamyl” refers to amino-carbonyl-amino (N—C(═O)—N) groups, in which the left amino is covalently bound to two moieties selected from hydrogen, alkyl, aryl, cycloalkyl, heterocycloalkyl, and heteroaryl and the right amino is covalently bound to one moiety selected from hydrogen, alkyl, aryl, cycloalkyl, heterocycloalkyl, and heteroaryl.

Alkyl, alkylene, alkenyl, alkynyl, cycloalkyl, heterocycloalkyl, cycloalkenyl, heterocycloalkenyl, aryl, and heteroaryl mentioned above can be substituted. Examples of substituents include alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, aryl, and heteroaryl.

The term “prodrug moiety” includes moieties which can be metabolized in vivo. Generally, the prodrugs moieties are metabolized in vivo by esterases or by other mechanisms to hydroxyl groups or other advantageous groups. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The structures of some of the tetracycline compounds of this invention include double bonds or asymmetric carbon atoms. Such compounds can occur as racemates, racemic mixtures, single enantiomers, individual diastereomers, diastereomeric mixtures, and cis- or trans- or E- or Z-double bond isomeric forms. Such isomers can be obtained in substantially pure form by classical separation techniques and by stereochemically controlled synthesis. Furthermore, the structures and other compounds and moieties discussed in this application also include all tautomers thereof.

The invention also pertains to a method for modulating, (e.g., increasing or decreasing) SMN2 mRNA splicing and SMNp levels. The method includes contacting the SMN2 mRNA with a tetracycline compound, such that SMN2 mRNA splicing is modulated. The modulation of SMN2 mRNA splicing by the tetracycline compounds of the invention may be determined by, for example, the cell-free splicing assay, the cellular gems assay, or by Western blot, RT-PCR assay in SMA patient fibroblasts grown in culture, RT-PCR analysis of cells or tissue after compound administration, Western blot for SMNp in cells or tissues treated with compound. In one embodiment, the tetracycline compound for modulating SMN2 mRNA splicing is not tetracycline. In another embodiment, the tetracycline compound increases cellular SMN protein levels in a subject. In yet another embodiment, the tetracycline compound increases gems in cells of said subject. One of skill in the art would understand that an increase in gems in cells may correlate with an increase in the cellular levels of SMN protein.

The terms “modulate,” “modulating” and “modulation” include increasing or decreasing SMN2 mRNA splicing or SMNp levels. The term “modulation of SMNp levels” includes the modulation of the expression of SMNp.

In one embodiment, the tetracycline compound increases the percentage of exon 7 inclusion and/or intron 6 during mRNA splicing. In another embodiment, the percentage of exon 7 inclusion during mRNA splicing may be determined by the assay described in Example 2. In yet another embodiment, the tetracycline compound increases the percentage of exon 7 inclusion during mRNA splicing by about 4-fold or greater, about 5-fold or greater, about 6-fold or greater, about 7-fold or greater, about 8-fold or greater, about 9-fold or greater, about 10-fold or greater, about 11-fold or greater, about 12-fold or greater, about 13-fold or greater, about 14-fold or greater, about 15-fold or greater, about 16-fold or greater, about 17-fold or greater, about 18-fold or greater, about 19-fold or greater, about 20-fold or greater, about 21-fold or greater, about 22-fold or greater, about 23-fold or greater, about 24-fold or greater, or about 25-fold. In one particular embodiment, the tetracycline compound increases the percentage of exon 7 inclusion during mRNA splicing of SMN2 by about 2.6-fold. In another embodiment, the tetracycline increases exon 7 inclusion by greater than 5-fold compared to background at a concentration of 10 μM. In yet another embodiment, the tetracycline compound increases exon 7 inclusion is about 19% compared to about 3% for a background at a concentration of 10 μM.

In one embodiment, the maximum percentage exon 7 inclusion observed upon administration of a tetracycline compound (e.g., Eₘₐₓ determined as described in Example 1) is at least about 5 percent or greater, at least about 10 percent or greater, at least about 15 percent or greater, at least about 20 percent or greater, at least about 25 percent or greater, at least about 30 percent or greater, at least about 35 percent or greater, at least about 40 percent or greater, at least about 45 percent or greater, at least about 50 percent or greater, at least about 55 percent or greater, at least about 60 percent or greater, at least about 65 percent or greater, at least about 70 percent or greater, at least about 75 percent or greater, at least about 80 percent or greater, at least about 85 percent or greater, at least about 90 percent or greater, at least about 95 percent or greater or at least about 100 percent. In a further embodiment, the maximum percentage exon 7 inclusion is at least about 23% or about 30%.

In another embodiment, the lowest concentration of a tetracycline compound at which maximum exon 7 inclusion is observed (e.g., Cₘₐₓ determined as described in Example 1) is less than about 30 μM, less than about 29 μM, less than about 28 μM, less than about 27 μM, less than about 26 μM, less than about 25 μM, less than about 24 μM, less than about 23 μM, less than about 22 μM, less than about 21 μM, less than about 20 μM, less than about 19 μM, less than about 18 μM, less than about 17 μM, less than about 16 μM, less than about 15 μM, less than about 14 μM, less than about 13 μM, less than about 12 μM, less than about 11 μM, less than about 10 μM, less than about 9 μM, less than about 8 μM, less than about 7 μM, less than about 6 μM, less than about 5 μM, less than about 4 μM, less than about 3 μM, less than about 2 μM or less than about 1 μM.

In one embodiment, the SMNp levels may be increased. In another embodiment, the SMNp levels in the subject may be increased by 1.8 fold or greater, about 2-fold or greater, about 3-fold or greater, about 4-fold or greater, about 5-fold or greater, about 6-fold or greater, about 7-fold or greater, about 8-fold or greater, about 9-fold or greater, about 10-fold or greater, about 11-fold or greater, about 12-fold or greater, about 13-fold or greater, about 14-fold or greater, about 15-fold or greater, about 16-fold or greater, about 17-fold or greater, about 18-fold or greater, about 19-fold or greater, about 20-fold or greater, about 21-fold or greater, about 22-fold or greater, about 23-fold or greater, about 24-fold or greater, about 25-fold or greater, about 26-fold or greater, about 27-fold or greater, about 28-fold or greater, about 29-fold or greater, about 30-fold or greater, about 31-fold or greater, about 32-fold or greater, about 33-fold or greater, about 34-fold or greater, about 35-fold or greater, about 36-fold or greater, about 37-fold or greater, about 38-fold or greater, about 39-fold or greater, about 40-fold or greater, about 41-fold or greater, about 42-fold or greater, about 43-fold or greater, about 44-fold or greater, about 45-fold or greater, about 46-fold or greater, about 47-fold or greater, about 48-fold or greater, about 49-fold or greater or about 50-fold or greater. In another embodiment, SMNp levels may be increased by about 18% in central nervous system tissue of which brain is one. In another embodiment, SMNp levels may be increased by about 40% in the brain.

Studies in a large cohort of SMA patients have revealed a tight inverted correlation between the amount of the protein encoded by the SMN2 gene and the clinical severity of the SMA. Thus, modulating SMN2 mRNA splicing to increase of full-length SMN protein levels can lead to treatment or prevention of spinal muscular atrophy. The present invention therefore also pertains to a method of treating or preventing a subject having spinal muscular atrophy. The method includes administering to the subject in need thereof an effective amount of a tetracycline compound, such that the SMA is treated or prevented. Advantageously, the tetracycline compounds used in the methods of the invention have one or more of the following characteristics: 1) potency in modulating mRNA splicing, 2) potency in modulating SMN protein levels, 3) central nervous system and/or brain penetration, 4) decreased phototoxic properties and 5) decreased antibacterial properties.

The term “spinal muscular atrophy” or “SMA” includes infantile SMA, SMA type 1 or Werdnig-Hoffman disease; intermediate SMA or SMA type 2; juvenile SMA, SMA type 3 or Kugelberg-Welander disease; and Adult SMA or SMA type 4.

The term “subject in need thereof” includes humans, and other animals, e.g., mammals (e.g., cats, dogs, horses, pigs, cows, sheep, rodents, rabbits, squirrels, bears, or primates) having spinal muscular atrophy or having an increased risk of developing spinal muscular atrophy. In one embodiment, the subject in need thereof is a human having spinal muscular atrophy.

The language “effective amount” of the tetracycline compound is that amount necessary or sufficient to treat or prevent SMAin a subject, e.g. prevent the various symptoms of SMA. The effective amount may vary depending on such factors as the size and weight of the subject, or the particular tetracycline compound. For example, the choice of the tetracycline compound may affect what constitutes an “effective amount.” One of ordinary skill in the art would be able to study the aforementioned factors and make the determination regarding the effective amount of the tetracycline compound without undue experimentation.

The regimen of administration may affect what constitutes an effective amount. The tetracycline compound may be administered to the subject either prior to or after the onset of SMA. Further, several divided dosages, as well as staggered dosages may be administered daily or sequentially, or the dose can be continuously infused, orally administered, administered by inhalation, or can be a bolus injection. The dosages of the tetracycline compound(s) may be proportionally increased or decreased as indicated by the exigencies of the therapeutic or prophylactic situation.

The term “treated,” “treating” or “treatment” describes the management and care of a patient for the purpose of combating a disease, condition, or disorder and includes the administration of an active agent of the present invention (e.g., the compound described above), or a pharmaceutically acceptable salt, prodrug, metabolite, polymorph or solvate thereof, to alleviate the symptoms or complications of a disease, condition or disorder, or to eliminate the disease, condition or disorder.

The term “preventing” or “prevent” as used herein includes either preventing the onset of a clinically evident disease progression altogether or preventing or slowing the onset of a preclinically evident stage of a disease in individuals at risk. This includes prophylactic treatment of those at risk of developing a disease.

The term “alleviate” or “ameliorate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Therapeutically effective dosages are expected to decrease the severity of a sign or symptom.

The term “symptom” is defined as an indication of disease, illness, injury, or that something is not right in the body. Symptoms are felt or noticed by the individual experiencing the symptom, but may not easily be noticed by others. Others are defined as non-health-care professionals.

The term “sign” is also defined as an indication that something is not right in the body. But signs are defined as things that can be seen by a doctor, nurse, or other health care professional.

The invention also relates to a pharmaceutical composition of a therapeutically effective amount of a compound of this invention (e.g., the exemplary compound shown above) and a pharmaceutically acceptable carrier. The invention also relates to a pharmaceutical composition of a therapeutically effective amount of a salt of a compound of this invention (e.g., the exemplary compound shown above) and a pharmaceutically acceptable carrier. The invention also relates to a pharmaceutical composition of a therapeutically effective amount of an N-oxide of a compound of this invention (e.g., the exemplary compound shown above) and a pharmaceutically acceptable carrier. The invention also relates to a pharmaceutical composition of a therapeutically effective amount of an N-oxide of salt of a compound of this invention (e.g., the exemplary compound shown above) and a pharmaceutically acceptable carrier. The invention also relates to a pharmaceutical composition of a therapeutically effective amount of a hydrate of a compound of this invention (e.g., the exemplary compound shown above) and a pharmaceutically acceptable carrier.

The method may further comprise administering the tetracycline compound in combination with a second agent, e.g., an agent which may enhance treatment of the spinal muscular atrophy.

The language “in combination with” a second agent includes co-administration of the tetracycline compound, and with the second agent, administration of the tetracycline compound first, followed by the second agent and administration of the second agent first, followed by the tetracycline compound. The second agent may be any agent which is known in the art to treat, prevent, or reduce the symptoms of a spinal muscular atrophy. Furthermore, the second agent may be any agent of benefit to the patient when administered in combination with the administration of a tetracycline compound. Examples of second agents include neuroprotective agents.

### Methods for Synthesizing Tetracycline Compounds of the Invention

The tetracycline compounds of the invention can be synthesized by using art recognized techniques, e.g., those shown in Scheme 1 below.

Scheme 1 outlines the general synthesis of 7-substituted phenyl tetracyclines. A 7-iodo sancycline derivative (1) may be reacted in a Stille coupling or a Suzuki coupling with an organotin derivative or a boronic acid derivative in the presence of a palladium catalyst to form a 7-substituted phenyl product (2).

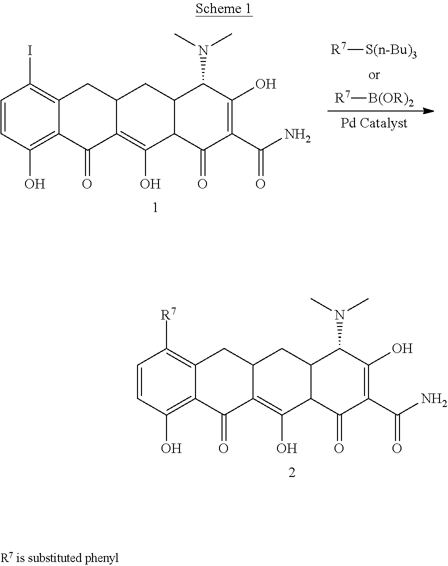

The reagents used in the above-described synthetic routes may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the desired tetracycline compounds. In addition, various synthetic steps may be performed in an alternate sequence or order to give the desired compounds. For example, compound (2) may be further modified via conventional chemical transformations to produce compounds of this invention. Synthetic chemistry transformations and protecting group methodologies (protection and deprotection) useful in synthesizing applicable indole compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The above scheme is only used for illustrative purposes. One skilled in the art, in view of this scheme and the examples provided herein, would appreciate that all of the compounds of this invention can be made by similar methods that are well known in the art.

The compounds thus obtained can be further purified by flash column chromatography, high performance liquid chromatography, crystallization, or any known purification method.

### Pharmaceutical Compositions for the Treatment of Spinal Muscular Atrophy

The invention also pertains at least in part to pharmaceutical compositions for the treatment of spinal muscular atrophy. The pharmaceutical compositions comprise a tetracycline compound of the invention in combination with a pharmaceutical acceptable carrier. The composition may further comprise a second agent for the treatment of spinal muscular atrophy or its symptoms. Each of the tetracycline compounds described herein may be used in pharmaceutical compositions of the invention.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, spinal, buccal, sublingual, rectal, vaginal, pulmonary and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane. Sprays also can be delivered by mechanical, electrical, or by other methods known in the art.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial, antiparasitic and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle. The compositions also may be formulated such that its elimination is retarded by methods known in the art.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration or administration via inhalation is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, administration via spinal tap, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually. Other methods for administration include via inhalation, intrathecal or intracerebroventricular injection or infusion.

The tetracycline compounds of the invention may also be administered to a subject via stents. The compounds may be administered through the stent or be impregnated in the stent itself.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats spinal muscular atrophy.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

As set out above, certain embodiments of the present compounds may contain a basic functional group, such as amino or alkylamino, and are, thus, capable of forming pharmaceutically acceptable salts with pharmaceutically acceptable acids. The term “pharmaceutically acceptable salts” is art recognized and includes relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Farm. SCI. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances includes relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl and propyl esters. (See, for example, Berge et al., supra.)

The invention also pertains, at least in part, to packaged compositions comprising the tetracycline compounds of the invention and instructions for using said compounds for the treatment of spinal muscular atrophy.

The compounds of this invention can be tested for their activity of modulating SMN2 mRNA splicing or the levels of SMNp in vitro or in vivo (see Example 2 below). The activity of the compounds in treating SMA can be assessed using an in vivo SMA animal model (e.g., mice). See the specific example below.

The invention is further illustrated by the following examples, which should not be construed as further limiting. The contents of all references, pending patent applications and published patents, cited throughout this application are hereby expressly incorporated by reference.

### EXAMPLE 1

### Synthesis of (4S,4aS,5aR,12aS)-4-dimethylamino-7-[3-(2-dimethylamino-ethylcarbamoyl)-4-fluoro-phenyl]-3,10,12,12a-tetrahydroxy-1,11-dioxo-1,4,4a,5,5a,6,11,12a-octahydro-naphthacene-2-carboxylic acid amide (Compound 1)

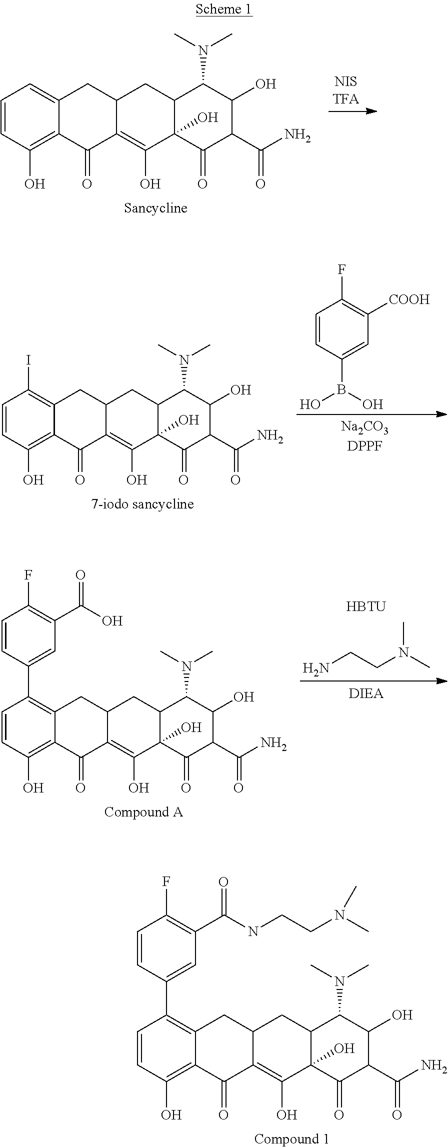

Preparation of 7-iodo sanscycline.TFA Salt

3.6 L of TFA was charged in a round bottom flask equipped with thermocouple, argon inlet and an overhead stirrer. 600 g of sanscycline hydrate was charged portion-wise in the flask and stirred for 15 min. The flask was placed in an ice/water and cooled to <8° C. 270 g of N-iodosuccinimide (NIS) was added into the reaction mixture over 5-10 minutes. The reaction mixture was stirred for 15-20 minutes, and then another 108 g of NIS was added. The mixture was allowed to warm to room temperature and stirred overnight. After HPLC confirmed the completion of reaction, the mixture was filtered through a bed of Celite and washed with 100 ml of TFA. The reaction mixture was concentrated to approximately ½ volume under reduced pressure. The resulting concentrate was poured into a mixture of 1.5 L of isopropanol and 13.5 L of EtOAc. The recovery flask was washed with 200 ml of EtOAc and added to the solution, then seeded with 7-iodo sancycline.TFA salt crystals. After stirring for 3-20 hours, the precipitate was filtered and washed with 3×500 ml of t-butyl methyl ether. The resulting material was dried to constant weight under reduced pressure. 835 g of 7-iodo sancycline.TFA salt (91% yield) was isolated in 96% of purity by HPLC.

### Preparation of Compound A

120 g of 7-iodo sancycline.TFA salt, 1.2 eq 3-carboxy 4-fluorobenzene boronic acid, 15% DPPF and 5 eq Na₂CO₃ were charged in a round bottom flask. Dioxane was added and the slurry was stirred for 20 min. Water was added portion-wise over 20 min at which point a clear solution was observed. Reaction was placed under vacuum and was degased with argon three times. The reaction was heated to 60-65° C. Reaction was complete in 2 hours. The reaction mixture was cooled to room temperature, filtered, and then precipitated in 3 volumes of acetonitrile. The resulting cake was washed twice with acetonitrile, and then dried under reduced pressure to a constant weight. 138 g of Compound A was isolated in 88% purity by HPLC.

### Preparation of Compound 1

100 g of Compound A was stirred in 700/300 mL of DMF/THF and was acidified using HCl to pH 1-2 to aid starting material dissolution. Once the starting material was dissolved, pH was adjusted to 6 with diisopropylethylamine and the mixture was cooled via an ice bath to 0-5° C. before 125 g of O-benzotriazole-N,N,N′,N′-tetramethyl-uronium-hexafluoro-phosphate (HBTU) was added. The reaction was stirred under 10° C. for 20 minutes. Then 100 mL (920 mmol) of dimethylaminoethyleneamine was added dropwise using an addition funnel and stirred for 90 min at room temperature resulting in a completed reaction by HPLC. The reaction solution was diluted with 1000 mL of isopropanol and then slowly added to 6000 mL of t-butyl methyl ether to precipitate the crude product. The resulting precipitate was filtered and rinsed twice with 2500 mL of t-butyl methyl ether and suck-dried under latex overnight to yield 153 g of sandy yellow powder to be purified by preparative chromatography. Prep. fractions were extracted with dichloromethane at pH 7.5 and concentrated to dryness. The dry concentrate was re-dissolved in MeOH and acidified to pH 1.1 with HCl and concentrated to dryness to yield Compound B.HCl salt (16.50 g, purity by HPLC =95.27%).

¹H-NMR (Bruker DPX300 300 MHz spectrometer, chemical shifts in ppm with TMS as internal reference at 0 ppm) δ 1.40-1.60 (m, 1H), 2.0-2.15 (m, 1H), 2.40-2.56 (m, 1H), 2.88-3.10 (m, 15H), 3.35-3.45 (m, 2H), 3.75-3.85 (m, 2H), 4.05 (s, 1H), 6.85-6.95 (m, 1H), 7.2-7.35 (m, 1H), 7.38-7.51 (m, 2H), 7.70-7.75 (m, 1H). MS (ESI) m/z 623 (M+H).

### EXAMPLE 2

### Biological Assays

In Vitro Correction of SMN2 mRNA Splicing in a Cell-Free System

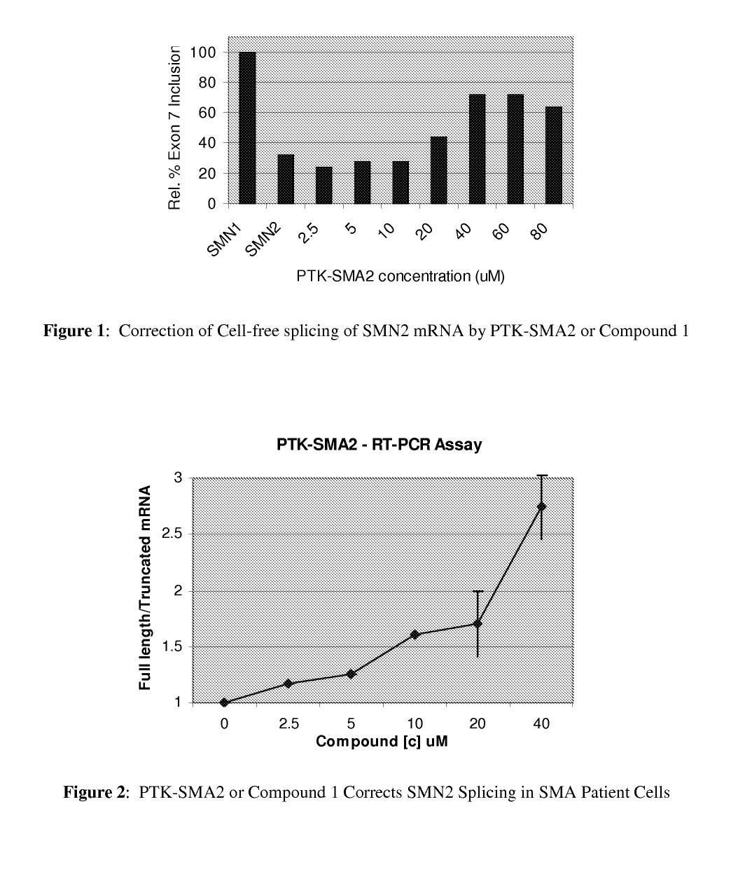

SMN2 pre-mRNA prepared in vitro from an SMN2 minigene containing only exons 6, 7 and the 5′ end of exon 8 and the intervening introns was incubated with the HeLa cell nuclear extract and Compound 1. The resulting spliced mRNA products were amplified by RT-PCR and separated by gel electrophoresis. The ratio of full-length to truncated SMN2 mRNA was determined. As shown in FIG. 1, Compound 1 successfully corrected the aberrant splicing of the SMN2 pre-mRNA in a dose dependent manner, and more than doubled the amount of full-length SMN2 mRNA produced from the pre-mRNA. These levels approached the amount of full-length mRNA produced from the SMN1 pre-mRNA added as a positive control.

### Correction of Splicing and Increases Functional SMN Protein in Type I SMA Patient Cells

SMA patient fibroblasts (cell line 3813) were incubated in the presence of Compound 1 for 24 hours and the levels of full-length and truncated SMN2 mRNAs were determined by RT-PCR and gel electrophoresis of cell extracts. As shown in FIG. 2, the compound increased the ratio of full-length to truncated SMN2 mRNA produced in the SMA patient cells in a dose-dependent manner. The increase observed corresponded to nearly tripling the amount of full-length SMN2 mRNA relative to the truncated form.

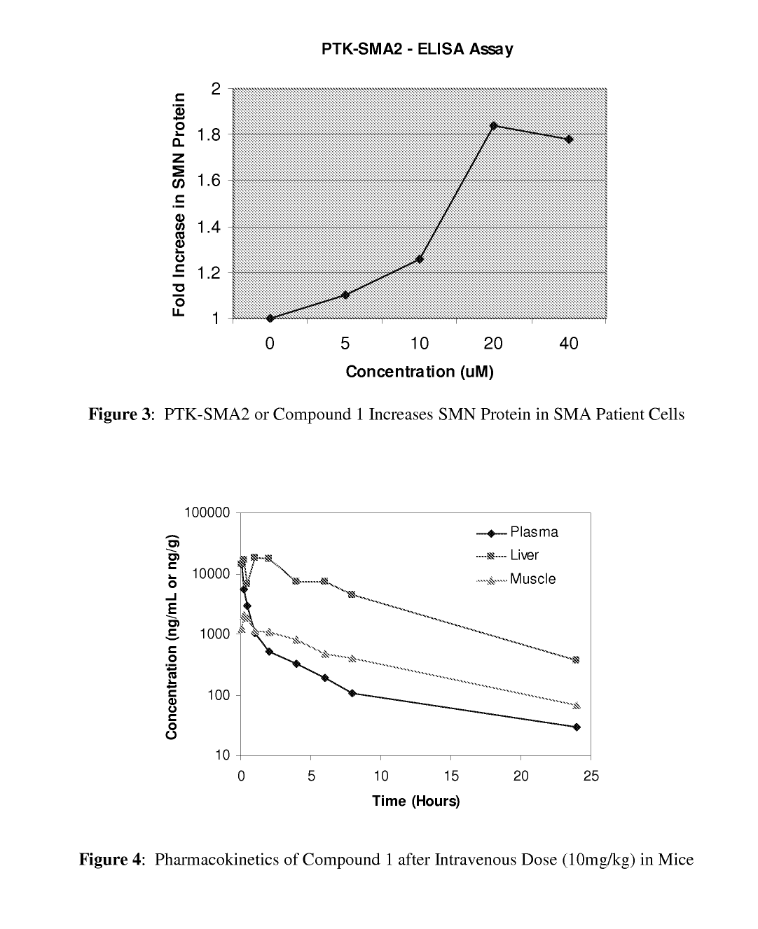

Using ELISA techniques, the cellular extracts from the treated patient cells were analyzed for SMN protein levels. FIG. 3 shows that the levels of the functional SMN protein in the SMA patient cells treated with 20 or 40 μM increased by 1.8 fold relative to that in the untreated cells.

### In Vivo Plasma, Tissue and CNS Exposure in Mice

Compound 1 was administered systemically and by continuous intracerebroventricular infusion to mice to evaluate the pharmacokinetics and preliminary safety of the compound. By the systemic route, Compound 1 was well-tolerated at all doses tested including a maximum daily dose of 50 mg/kg for 8 days. After a single intravenous dose of 10 mg/kg, the concentration of Compound 1 in plasma reached the level tested in the SMA patient cell assay and remained detectable in plasma for 24 hours. Significant tissue penetration was observed in the muscle and liver with compound levels remaining 3 and 20-fold higher than plasma at 24 hours, respectively. These results are shown in FIG. 4. Compound 1 was also detected in the brain after systemic dosing at levels approximately 22% those in plasma. See Table 1.

TABLE 1
Brain Levels of Compound 1 after Intravenous
Dose (10 mg/kg) in Mice

| | ng/mL or ng/g | | |
|---|---|---|---|
| Tissue | 0.5 hr | 1 hr | 3 hr |
| Plasma | 3100 | 900 | 400 |
| Brain | 550 | 210 | 110 |
| Brain/Plasma (%) | 18% | 23% | 28% |

Direct dosing to the CNS of mice was well tolerated for 6 days of continuous pump infusion (8 mg/kg) and levels of Compound 1 reached concentrations well in excess of those tested in the SMA patient cell assay. The mean concentration in the mouse brain at day 6 was 204 μM (or 127 μg/mL) compared to activity at 10-20 μM in patient cells, see FIGS. 2 and 3.

Correction of SMN2 mRNA Splicing and Increase of SMN Protein Levels in Mice Containing the Human SMN2 Transgene (hSMN2)

A series of in vivo experiments were performed using adult transgenic mice which carry and express the human SMN2 gene (mice known as the original Burghes strain). See Monani, U. R., et al., The human centromeric survival motor neuron gene (SMN2) rescues embryonic lethality in Smn (−/−) mice and results in a mouse with spinal muscular atrophy. 2000. Hum. Mol. Genet., 9(3): 333-9. In adult hSMN2 transgenic mice, Compound 1 was dosed by intracerebroventricular (i.c.v.) infusion into the right ventricle for 6 days. It was observed that splicing of SMN2 pre-mRNA in the brain tissue of the mice was significantly corrected, increasing the inclusion of exon 7 from less than 10% in the untreated animals, to more than 60%—a more than 600% increase in the ratio of full-length to truncated mRNA. See Table 2. A significant increase of hSMN protein in the brain tissue was also observed −57% increase over untreated control animals.

Systemic dosing (intraperitoneal, i.p.) of Compound 1 for 8 days in the same SMN2 transgenic mouse strain also enhanced hSMN protein levels. In the liver tissue, SMN protein levels increased 165% in treated mice over untreated controls. In the brains, SMN protein levels increased in the mice treated systemically with Compound 1 by 18-44%. See also Table 2.

TABLE 2
Correction of Splicing and Increases SMN
Protein in Adult SMN2 Transgenic Mice

| | | | SMN2 mRNA | SMN Protein |
|---|---|---|---|---|
| Route | Tissue | Dose | % Change* | % Change† |
| ICV | Brain | 8 mg/kg | +648% | +57% |
| IP | Brain | 25 mg/kg | No change | +44% |
| | | 50 mg/kg | No change | +18% |
| | Liver | 25 mg/kg | +32% | +75% |
| | | 50 mg/kg | +6% | +165% |

*% change of SMN2 mRNA full-length/truncated ratio.
†Percent increase of hSMN protein relative to actin.
ICV = intracerebroventricular infusion; IP = intraperitoneal injection.

(4S,4aS,5aR,12aS)-4-Dimethylamino-7-[3-(2-dimethylaminomethyl)-4-methoxy-phenyl]-3,10,12,12a-tetrahydroxy-1,11-dioxo-1,4,4a,5,5a,6,11,12a-octahydro-naphthacene-2-carboxylic acid amide was also tested in this assay. The results show that at the dose of 8 mg/kg/day, the ratio of SMN mRNA full-length/truncated was 83% and the increase of hSMN protein was 11%. The concentration of this compound in the brain is 100 μM (compared with 200 μM for Compound 1) and the ratio of the concentration in the brain and the concentration in the plasma is 8% (compared with 29% for Compound 1).

### Toleration and Correction of SMN2 Splicing in Neonatal SMN2 Transgenic Mice

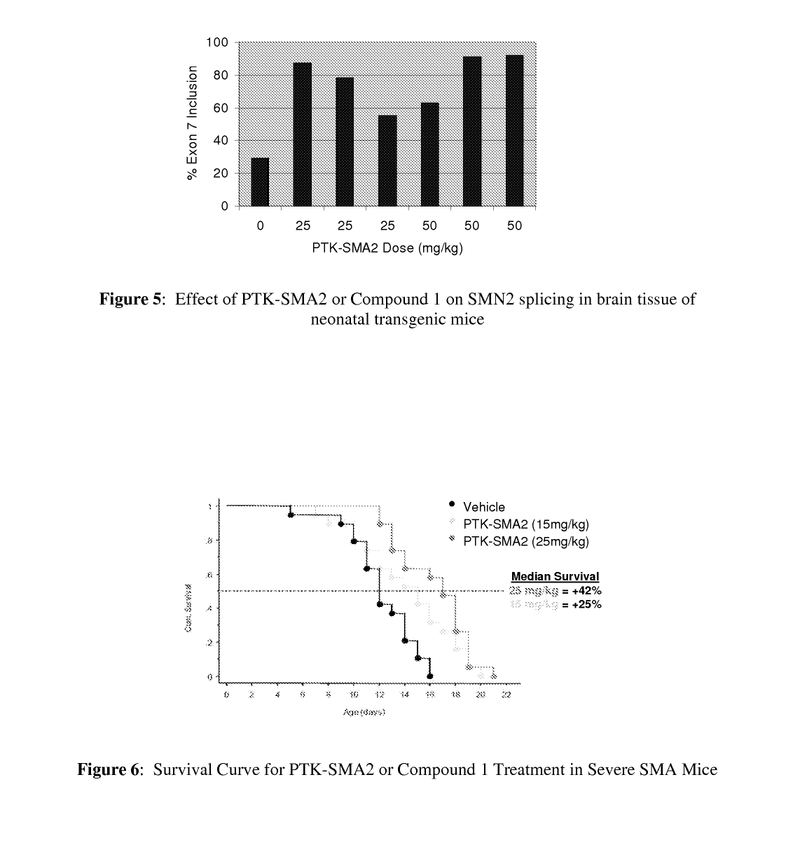

Compound 1 was tested for safety and the ability to correct SMN2 splicing in neonatal mice containing the SMN2 and SMN2Δ7 transgenes. See Le, T. T., et al., Hum. Mol. Genet., 2005, 14(6): 845-57. Wild type hSMN2;SMN2Δ7 mice were dosed intraperitoneally with 25 or 50 mg/kg of Compound 1 starting on day 3 after birth and continuing for 8 days. The compound was well tolerated at both doses and significantly corrected the splicing of SMN2 mRNA at both doses in the brains of the treated mice. RT-PCR of the SMN2 mRNA in brain tissue revealed that Compound 1 increased inclusion of exon 7 from 29% in untreated animals to as high as 92% in the high dose group. The results are showin in FIG. 5.

### Improved Survival in a Mouse Model of Severe SMA

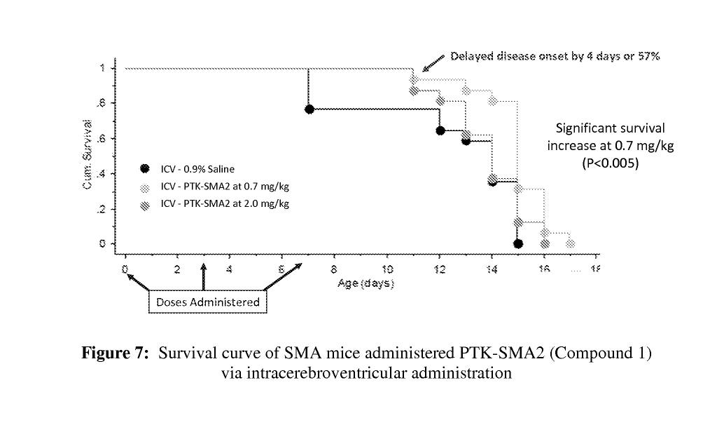

Compound 1 was tested for the ability to extend survival and improve motor function in a mouse model of severe Type I SMA. Neonatal mice that lack the mouse SMN gene but contain the human SMN2 and SMN2Δ7 transgenes were used. These mice typically develop SMA-like disease shortly after birth and live for an average of 13 days before succumbing to the disease. Compound 1 was administered via the intraperitoneal route starting at day 3 after birth and continued daily until death. As shown in FIG. 6, at a dose of 25 mg/kg, Compound 1 significantly improved survival of the SMA mice with an increase of 42% or 6 days over untreated control animals. At the low dose of 15 mg/kg, a statistical increase in survival was also observed (increase in survival time by 25%). Also, improvements in motor function and body weight were noted in the treated mice. Compound 1 was also administered by intracerebroventricular injections to SMA mice on the day of birth (P0), then day 3 and finally day 7 after birth. Doses of 2.0 mg/kg and 0.7 mg/kg were given and survival and motor phenotype were monitored. Both doses delayed disease onset by 4 days and 0.7 mg/kg significantly improved median survival by 7% (FIG. 7). Improvements in motor function and body weight were also noted.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

All patents, patent applications, and literature references cited herein are hereby expressly incorporated by reference.

1. A compound of formula (I) or a pharmaceutically acceptable salt thereof: 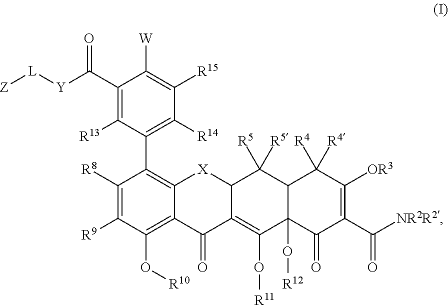
wherein
X is CR⁶R^{6′}, C═CR⁶R^{6′}, NR^{6″}, O, or S;
each of R², R^{2′}, and R^{6″}, independently, is hydrogen, halo, or Rⁱ; Rⁱ being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkoxy, C₁-C₆ alkylcarbonyl, arylcarbonyl, C₁-C₆ alkylsulfinyl, arylsulfinyl, C₁-C₆ alkylsulfonyl, arylsulfonyl, or arylalkyl;
each of R³, R¹⁰, R¹¹ and R¹², independently, is hydrogen or Rⁱⁱ; Rⁱⁱ being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkylcarbonyl, arylcarbonyl, C₁-C₆ alkylsulfinyl, arylsulfinyl, C₁-C₆ alkylsulfonyl, arylsulfonyl, arylalkyl, or a prodrug moiety;
each of R⁴, R^{4′}, R⁵, R^{5′}, R⁶, R^{6′}, R⁸, R⁹, R¹³, R¹⁴, and R¹⁵, independently, is hydrogen, halo, nitro, cyano, hydroxyl, thiol, or Rⁱⁱⁱ; Rⁱⁱⁱ being C₁-C₆ alkyl, C₂-C₆ alkenyl, C₂-C₆ alkynyl, aryl, heteroaryl, C₃-C₈ cycloalkyl, 3 to 8-membered heterocycloalkyl, arylalkyl, C₁-C₆ alkyloxy, C₃-C₈ cycloalkyloxy, 3 to 8-membered heterocycloalkyloxy, aryloxy, heteroaryloxy, C₁-C₆ alkylthio, arylthio, C₁-C₆ alkylsulfinyl, arylsulfinyl, C₁-C₆ alkylsulfonyl, arylsulfonyl, C₁-C₆ alkylamino, arylamino, di-C₁-C₆ alkylamino, diarylamino, C₁-C₆ alkylcarbonyl, C₃-C₈ cycloalkylcarbonyl, 3 to 8-membered heterocycloalkylcarbonyl, arylcarbonyl, heteroarylcarbonyl, C₁-C₆ alkylcarboxyl, C₃-C₈ cycloalkylcarboxyl, 3 to 8-membered heterocycloalkylcarboxyl, arylcarboxyl, heteroarylcarboxyl, C₁-C₆ alkyloxycarbonyl, C₃-C₈ cycloalkyloxycarbonyl, 3 to 8-membered heterocycloalkyloxycarbonyl, aryloxycarbonyl, heteroaryloxycarbonyl, C₁-C₆ alkylcarbamido, C₃-C₈ cycloalkylcarbamido, 3 to 8-membered heterocycloalkylcarbamido, arylcarbamido, heteroarylcarbamido, C₁-C₆ alkylcarbamyl, C₃-C₈ cycloalkylcarbamyl, 3 to 8-membered heterocycloalkylcarbamyl, arylcarbamyl, or heteroarylcarbamyl;
W is halo, alkyl, amino, cyano, nitro, C₁-C₆ alkoxy, hydroxyl, or thiol;
Y is O, or NR^{7a};
L is C₁₋₆ alkylene;
Z is OR^{7b}, SR^{7b}, or NR^{7b}R^{7c}; and
each of R^{7a}, R^{7b}, and R^{7c}, independently, is hydrogen, halo, or R^{iv}, R^{iv} being C₁-C₆ alkyl, C₂-C₆ alkenyl, C₂-C₆ alkynyl, C₃-C₈ cycloalkyl, 3 to 8-membered heterocycloalkyl, aryl, heteroaryl, C₁-C₆ alkoxy, C₁-C₆ alkylcarbonyl, arylcarbonyl, C₁-C₆ alkylsulfinyl, arylsulfinyl, C₁-C₆ alkylsulfonyl, arylsulfonyl, or arylalkyl;
wherein each of Rⁱ, Rⁱⁱ, Rⁱⁱⁱ, and R^{iv} is optionally substituted with one or more substituents selected from the group consisting of halogen, cyano, nitro, hydroxyl, amino, thiol, C₁-C₆ alkyl, C₂-C₆ alkenyl, C₂-C₆ alkynyl, aryl, heteroaryl, C₃-C₈ cycloalkyl, 3 to 8-membered heterocycloalkyl, arylalkyl, C₁-C₆ alkyloxy, C₃-C₈ cycloalkyloxy, 3 to 8-membered heterocycloalkyloxy, aryloxy, heteroaryloxy, C₁-C₆ alkylthio, arylthio, C₁-C₆ alkylsulfinyl, arylsulfinyl, C₁-C₆ alkylsulfonyl, arylsulfonyl, C₁-C₆ alkylamino, arylamino, di-C₁-C₆ alkylamino, diarylamino, C₁-C₆ alkylcarbonyl, C₃-C₈ cycloalkylcarbonyl, 3 to 8-membered heterocycloalkylcarbonyl, arylcarbonyl, heteroarylcarbonyl, C₁-C₆ alkylcarboxyl, C₃-C₈ cycloalkylcarboxyl, 3 to 8-membered heterocycloalkylcarboxyl, arylcarboxyl, heteroarylcarboxyl, C₁-C₆ alkyloxycarbonyl, C₃-C₈ cycloalkyloxycarbonyl, 3 to 8-membered heterocycloalkyloxycarbonyl, aryloxycarbonyl, heteroaryloxycarbonyl, C₁-C₆ alkylcarbamido, C₃-C₈ cycloalkylcarbamido, 3 to 8-membered heterocycloalkylcarbamido, arylcarbamido, heteroarylcarbamido, C₁-C₆ alkylcarbamyl, C₃-C₈ cycloalkylcarbamyl, 3 to 8-membered heterocycloalkylcarbamyl, arylcarbamyl, and heteroarylcarbamyl.

2. The compound of claim 1, wherein X is CR^{6′}R⁶; R⁴ is NR^{4a}R^{4b}; R^{4a} and R^{4b} are each C₁-C₆ alkyl, and R², R^{2′}, R³, R^{4′}, R⁵, R^{5′}, R⁶, R^{6′}, R⁸, R⁹, R¹⁰, R¹¹, R¹², R¹³, R¹⁴, and R¹⁵ are each hydrogen.

3. The compound of claim 2, wherein Y is NR^{7a}, R^{a} being H.

4. The compound of claim 3, wherein L is C₂₋₃ alkylene.

5. The compound of claim 4, wherein L is —CH₂CH₂—.

6. The compound of claim 5, wherein Z is OR^{7b}, R^{7b} being C₁-C₆ alkyl.

7. The compound of claim 5, wherein Z is NR^{7b}R^{7c}, each of R^{7b} and R^{7c}, independently, being hydrogen or C₁-C₆ alkyl.

8. The compound of claim 7, wherein each of R^{7b} and R^{7c} is methyl.

9. The compound of claim 8, wherein W is F, Cl, or I.

10. The compound of claim 9, wherein W is F.

11. The compound of claim 1, wherein W is F, Cl, or I.

12. The compound of claim 11, wherein W is F.

13. The compound of claim 12, wherein Y is NR^{7a}, R^{a} being H.

14. The compound of claim 13, wherein L is C₂₋₃ alkylene.

15. The compound of claim 14, wherein L is —CH₂CH₂—.

16. The compound of claim 15, wherein Z is OR^{7b}, R^{7b} being C₁-C₆ alkyl.

17. The compound of claim 15, wherein Z is NR^{7b}R^{7c}, each of R^{7b} and R^{7c}, independently, being hydrogen or C₁-C₆ alkyl.

18. The compound of claim 17, wherein each of R^{7b} and R^{7c} is methyl.

19. The compound of claim 1, wherein Y is NR^{7a}, R^{a} being H.

20. The compound of claim 19, wherein L is C₂₋₃ alkylene.

21. The compound of claim 20, wherein L is —CH₂CH₂—.

22. The compound of claim 21, wherein Z is OR^{7b}, R^{7b} being C₁-C₆ alkyl.

23. The compound of claim 21, wherein Z is NR^{7b}R^{7c}, each of R^{7b} and R^{7c}, independently, being hydrogen or C₁-C₆ alkyl.

24. The compound of claim 1, wherein the compound is:

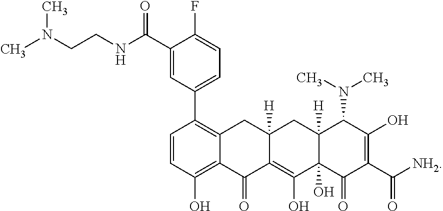 [SMILES: CN(C)CCNC(=O)c1cc(-c2ccc(O)c3c2C[C@H]2C[C@H]4[C@H](N(C)C)C(O)=C(C(N)=O)C(=O)[C@@]4(O)C(O)=C2C3=O)ccc1F]

25. A pharmaceutical composition comprising a compound of claim 1 and a pharmaceutically acceptable carrier.

26. A method of treating or preventing spinal muscular atrophy, comprising administering to a subject in need thereof an effectively amount of a compound of claim 1.
